(19) United States
(12) Patent Application Publication
(10) Pub. No.: US 2005/0261701 A1
(43) Pub. Date: Nov. 24, 2005

(54) Surgical device for a anterolateral reconstruction

(76) Inventors: David A. McGuire, Anchorage, AK (US); Jeffrey C. Wolchok, Anchorage, AK (US); Steven D. Hendricks, Anchorage, AK (US)

Correspondence Address: BROMBERG & SUNSTEIN LLP, 125 SUMMER STREET, BOSTON, MA, 02110-1618, US

(21) Appl. No.: 11/125,808

(22) Filed: May 10, 2005

Related U.S. Application Data
(60) Provisional application No. 60/569,987, filed on May 11, 2004.

(51) Int. Cl. A61B 17/58

(52) U.S. Cl. 606/96

(57) ABSTRACT

An isometer for determining isometric graft tunnel placement for lateral side surgical augmentation or reconstruction for anterolateral rotatory knee instability. This graft tunnel placement isometry assessment and measurement device is designed to be used with a graft and an extra-articular technique that restores rotatory, lateral, and anterior knee stability when used by itself or in combination with intra-articular ACL reconstruction. The isometer includes an elongated body with a slider rod that slides horizontally within the body and a cannulated fixed stylus attached transversely to the first end of the elongated body. Within that cannulated fixed stylus is a guide pin that slides freely but that can also be fixed using a fixed-stylus set screw. Similarly, a cannulated movable stylus is attached transversely to the first end of the slider rod, and within that cannulated movable stylus is a guide pin that slides freely but that can also be fixed using a movable-stylus set screw. There is a pointer encircling the slider rod that slides with the slider rod, but whose position along the slider rod can also be adjusted manually. To determine rotary knee isometry during knee reconstructive surgery, the guide pins are positioned over previously identified lateral reconstruction tunnel sites. The guide pins are then driven into the bone tunnel sites and the knee put through a full range of motion by flexing and extending the knee while the isometer position is maintained and the pointer movement is monitored. If movement of the pointer is unacceptably large, the guide pins are re-set, re-positioned and re-driven into different pre-identified lateral reconstruction tunnel sites, and pointer movement during full range motion is again monitored until movement is acceptably small.

### PRIORITY

The present U.S. patent application claims priority from U.S. provisional patent application, Serial No. 60/569,987, filed on May 11, 2004 entitled “Surgical Device for Anterolateral Reconstruction” which is incorporated by reference herein in its entirety.

### BACKGROUND OF THE INVENTION

The present invention relates to surgical devices and procedures used in the field of knee surgery, particularly anterolateral reconstruction in combination with reconstruction of the anterior cruciate ligament (ACL) of the knee.

Three situations have been identified that frequently lead to severe anterolateral rotary (ALR) instability in the knee. In such cases, anterolateral reconstruction should be considered in combination with ACL reconstruction. The three situations are: 1) when patients present with a failed isolated ACL reconstruction in which the tunnels, the graft and the rehabilitation all appear to have been properly done; 2) when patients who have had a previous lateral reconstruction in conjunction with an ACL reconstruction are persistently unstable; and 3) when patients have suffered a knee dislocation. Other situations may exist, and should be determined on an individual basis.

Failure to recognize such severe anterolateral rotary instability and to perform the appropriate extra-articular reconstruction is an under-recognized cause of ACL reconstruction failure. Thus, a means for identifying prospective ACL reconstruction failure candidates is needed, for example, a mechanism for assessing lateral rotary instability/stability.

The Lachman's and flexion rotation drawer tests can be used to assist in the diagnoses of severe lateral rotary instability, helping to identify candidates that should be considered for anterolateral reconstruction in combination with the ACL reconstruction. Those patients with positive Lachman's and flexion rotation drawer tests with grades of at least 2+ have proved to be grossly unstable. Once identified, a procedure and/or device for determining and restoring lateral isometry within an acceptable range during ALR reconstruction is needed. Presently, surgeons rely on “free hand” techniques to determine and restore lateral isometry during ALR reconstructions. These “free hand” methods are time consuming and not always accurate. A faster, more reliable and more reproducible method and apparatus for determining and restoring lateral isometry would thus benefit both surgeons and patients.

### SUMMARY OF THE INVENTION

The present invention as embodied is directed to a surgical device and method of using the surgical device, referred to as an isometer, for restoring rotary and anterior knee stability when used in combination with an intra-articular ACL reconstruction. The isometer eliminates the variables in “free hand” determination and restoration of lateral rotary stability, and also decreases the time needed to determine lateral rotary isometry.

The isometer includes an elongated body with a slider rod that slides horizontally within the body along a longitudinal slot running nearly the length of the rod. A cannulated fixed stylus is attached transversely to the first end of the elongated body and within that cannulated fixed stylus is a fixed-stylus guide pin that slides freely and but that can be fixed using a fixed-stylus set screw. A cannulated movable stylus is attached transversely to the first end of the slider rod, and within that cannulated movable stylus is a movable-stylus guide pin that slides freely within the cannulated movable stylus but that can be fixed using a movable-stylus set screw. There is a pointer encircling the slider rod that slides with the slider rod, but whose position along the slider rod can also be adjusted manually.

In use, the guide pins are directed into the fixed and movable styli, advanced until the tips protrude beyond the fixed and movable stylus ends, and then immobilized within the styli by tightening the set screws. Once immobilized within the fixed and movable styli, the immobilized guide pins are positioned over previously identified lateral reconstruction tunnel sites such that the fixed stylus and guide pin are positioned over a femoral tunnel site and the movable stylus and guide pin are positioned over a tibial tunnel site. Of course, it is understood that the placement site of pin positioning may be reversed, with the immobilized stylus and guide pins being positioned over a femoral tunnel site and the movable stylus and guide pins being positioned over a tibial tunnel site.

The guide pins are next driven into the bone tunnel sites. In another variation, the guide pins may be first driven into the bone tunnel sites, and then the isometer seated on the guide pins. After the pins are inserted into the bone tunnel sites, and the pins are locked into position using a locking mechanism, such as the previously described set screws, the knee is put through a full range of motion by flexing and extending the knee while the isometer position is maintained. During these full range movements, the movement of the pointer in relation to the isometer body is monitored. If movement of the pointer is unacceptably large (greater than 2 mm, 2.5 mm or 3 mm), the fixed, movable, or both guide pins are re-set, re-positioned and re-driven into different pre-identified lateral reconstruction tunnel sites and the movement of the pointer during full range motion is again monitored until the pointer movement is acceptably small. The determination of the maximum distance allowable is surgeon dependent and depends on the patient's anatomy. In ideal or near ideal circumstances the maximum movement would be no greater than 2 mm.

At this time, the set screws for the fixed and movable guide pins within their respective styli are loosened and the isometer is removed leaving the guide pins in place for completion of the reconstructive surgery. Once the isometer, is removed from the guide pins, a cannulated drill can be seated on a guide pin and a femoral tunnel can be drilled. Similarly, the cannulated drill can be seated on the other guide pin and a tibial tunnel can be drilled.

The isometer of the present invention substantially eliminates the guess-work and skill-intensive techniques inherent in “free-hand” isometry determinations during ALR/ACL combination reconstructive knee surgery, and greatly standardizes the process for such determinations.

### BRIEF DESCRIPTION OF THE DRAWINGS

FIG. 1 illustrates an isometer device of the present invention;

FIG. 2 illustrates an enlarged view of the elongated isometer body;

FIG. 3A illustrates a top view of the slider rod;

FIG. 3B illustrates a side view of the slider rod;

FIG. 4 is a flow chart showing the steps for using the isometer;

FIG. 5 shows a bone model with proposed placement of the tibial and femoral tunnels;

FIG. 6 shows the bone model of FIG. 5 superimposed on a knee during surgery; and

FIG. 7 shows the placement of the isometer on the knee for determining if there is isometry for the tunnel sites.

### DETAILED DESCRIPTION OF THE INVENTION

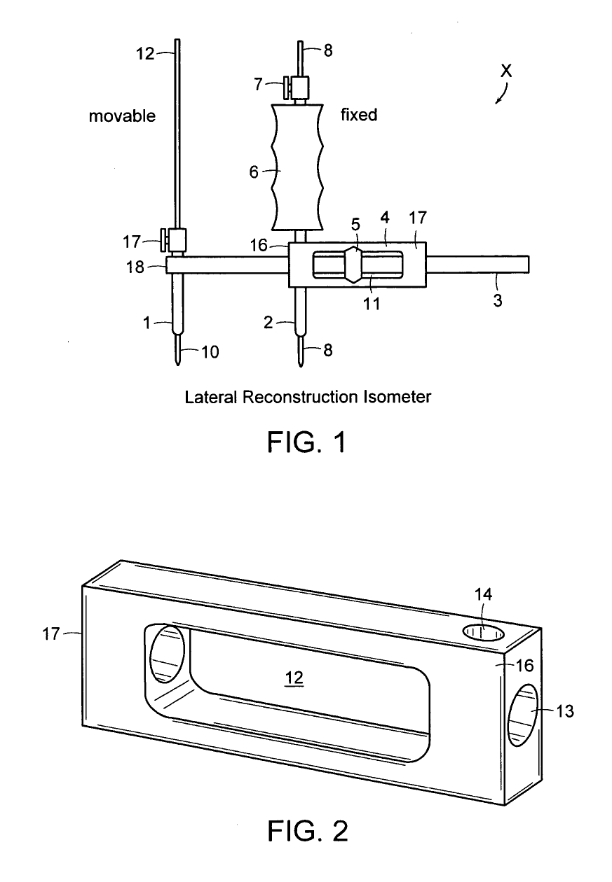

Referring now to FIG. 1, an isometer X having an elongated cannulated body 4, having a first end 16 and a second end 17, that encases a slider rod 3 with a pointer 5, further having a fixed stylus 2 and corresponding guide pin 8, and a movable stylus 1 and corresponding guide pin 10, is designed for use in determining lateral isometry for restoring rotary and anterior knee stability in combination with ACL reconstruction.

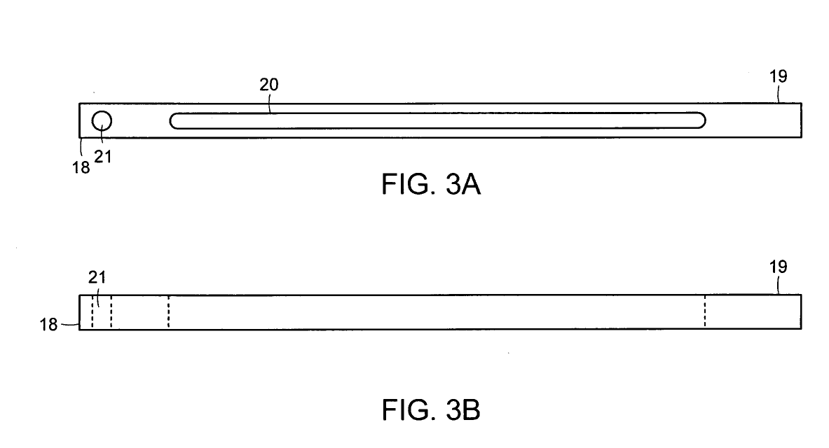

The slider rod 3 has a first end 18 and second end 19 and a longitudinal slot 20 (see FIG. 3A for slot 20) running nearly the length of the rod, and slides horizontally within the hollow elongated body 4. There is also a hole 21 (see FIG. 3B) in first end 18 wherein the movable-stylus guide pin 10 slides vertically within the cannulated movable stylus 1, and is attached transversely through the first end 18 of the slider rod 3. Encircling the slider rod 3 is a pointer 5 that slides with the slider rod 3 but whose position along the slider rod 3 may also be adjusted manually. The slider rod may also be threaded on the first end to receive the movable stylus 1.

The cannulated fixed stylus 2 is attached transversely through the first end 16 of the isometer body 4 and has a corresponding fixed-stylus guide pin 8 that slides vertically within the cannulated fixed stylus 2, through the first end 16 of the isometer body 4. A fixed-stylus set screw 7 is attached near the open-end top of the fixed stylus 2 for immobilizing and releasing the fixed-stylus guide pin 8 within the fixed stylus 2.

The cannulated movable stylus 1 is attached transversely through the first end 18 of the slider rod 3 and has a corresponding movable-stylus guide pin 10 that slides vertically within the cannulated movable stylus 1, through the first end 18 of the slider rod 3. A movable-stylus set screw 9 is attached near the open-end top of the movable stylus 1 for immobilizing and releasing the movable-stylus guide pin 10 within the movable stylus 2.

There may also be a cannulated handle 6 attached to the isometer body 4, aligned in such a way that the fixed-stylus guide pin 8 can be directed through the handle cannulation, though the isometer body 4, and through the slider rod 3, and through the fixed stylus 2.

The cannulated elongated body may also have a side window opening 12 for viewing movement of the pointer 5 along the slider rod 3.

During intra-articular ACL reconstruction, first a lateral incision is made through the subcutaneous tissue from the lateral femoral epicondyle to Gerdy's Tubercle. The fascia lata is divided longitudinally. Gerdy's tubercle is identified along with the site just posterior to the insertion of the lateral collateral ligament on the femur as the sites of the reconstruction tunnels as shown in FIG. 1A. The isometer is then used.

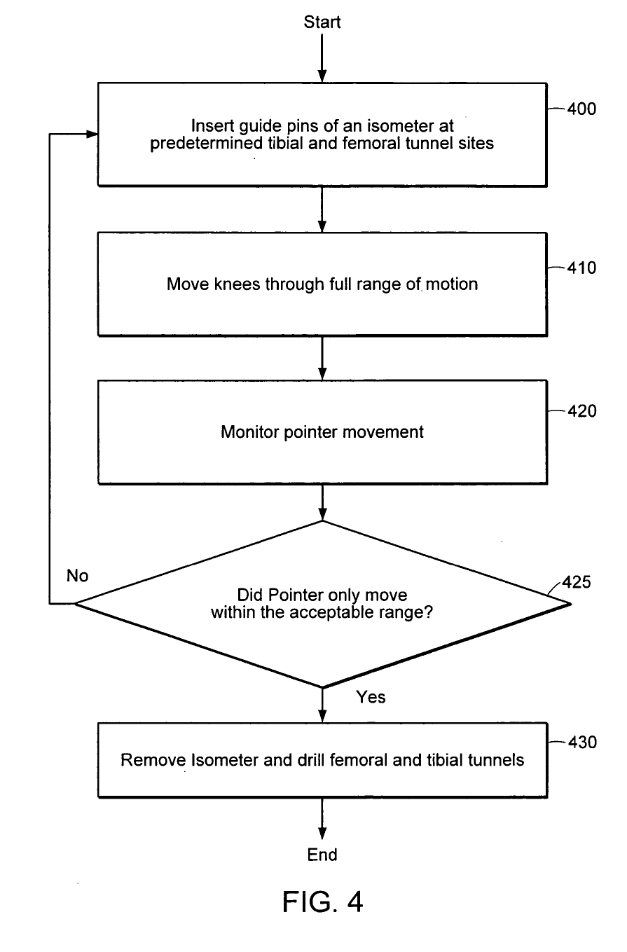

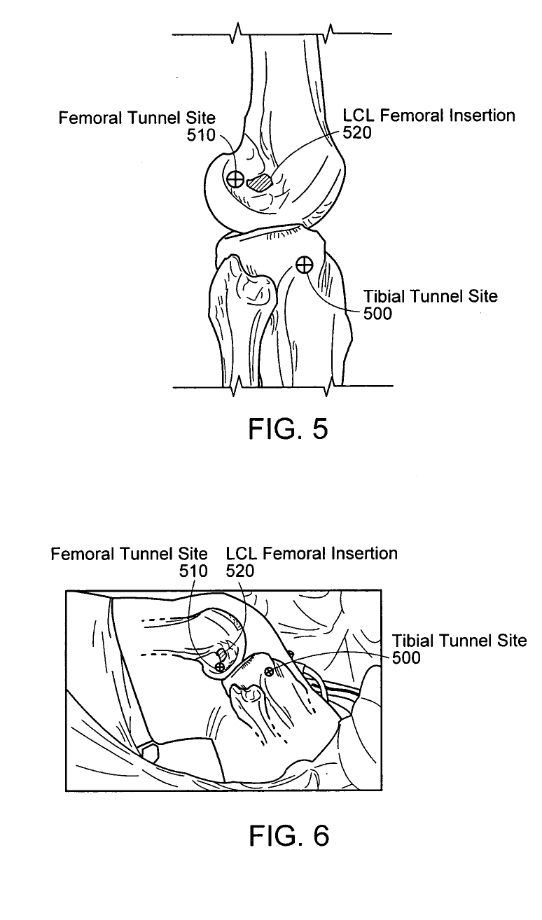

FIG. 4 provides a flow chart of the method of using the isometer to determine a position of isometry for restoring lateral stability during ACL reconstruction. First, the fixed- and movable-stylus guide pins 8 and 10 are initially directed into the corresponding fixed and movable styli 2 and 1 and advanced until the guide pins protrude beyond the corresponding stylus tips preferably ¼ inch. The guide pins 8 and 10 are then immobilized within the corresponding styli 2 and 1 by tightening the corresponding set screws 7 and 9. The immobilized guide pins 8 and 10 are then positioned over previously identified lateral reconstruction tunnel sites through skin incisions such that the fixed stylus 2 and its guide pin 8 are positioned over a femoral tunnel site and the movable stylus 1 and its guide pin 10 are positioned over a tibial tunnel site (Step 400). The initial tunnel placement sites are identified as Gerdy's tubercle on the tibia 500 along with the site 510 just posterior to the insertion of the lateral collateral ligament 520 on the femur as shown in FIG. 5 and FIG. 6.

The placement sites of the stylus and guide pins may be reversed, such that the fixed stylus 2 and its guide pin 8 are positioned over previously identified tibial tunnel sites and the movable stylus 1 and its guide pin 10 are positioned over previously identified femoral tunnel sites.

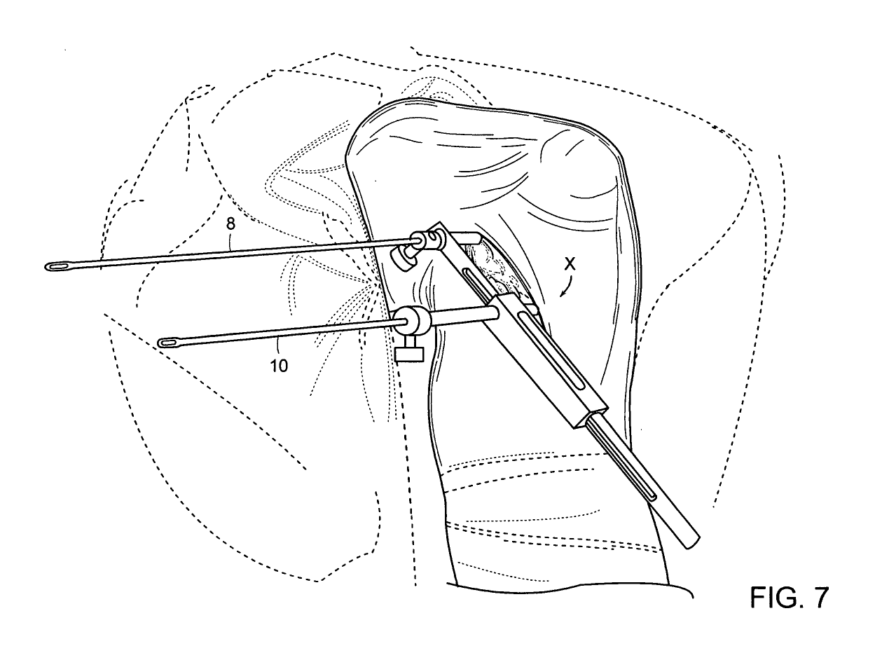

The guide pins 8 and 10 are then driven into the bone tunnel sites, preferably using a light mallet. The pins are driven preferably to a depth of about ⅛ to ¼ inch. In another embodiment, the pins are first driven into the bone and then the isometer is placed on the pins. FIG. 7 shows the guide pins 8 and 10 in place during surgery with the isometer X positioned over the guide pins. The isometer position is maintained as the knee is then flexed and extended slowly throughout its full range of motion in the anterior-posterior axis in the sagittal plane. (Step 410) This test of the range of motion is used to find a substantially isometric point. If the points are not substantially isometric, the graft will be too lose in one area of knee flexion or too tight in another area of flexion. If the graft is too tight the knee will either not be able to move fully or the graft will stretch. During this test of the full movement of knee motion, the movement of pointer 5 is monitored in relation to the isometer body 4 (Step 420). The isometer body may be provided with markings which are indicative of distance. The surgeon then access whether the movement of the pointer is acceptable (Step 425). A pointer movement maximum of less than 2 mm is preferred and a value of between 2 mm and 3 mm is an indication that the positions of the pin are approximately at isometric locations. As a result, the markings on the isometer body are preferably in increments of 1 mm or less. Further, markings which indicate the boundary of acceptable range of movement may be provided in place of or in addition to the markings which are indicative of distance. If the movement of the pointer is acceptable, the isometer is removed as explained below, leaving the guide pins. A cannulated drill is then seated on the guide pins and the tibial and femoral tunnels are drilled (Step 430). If the movement of pointer 5 is not acceptable, for example more than 3 mm, the movable-stylus guide pin 10 and/or fixed-stylus guide pin 8 is released by loosening corresponding set screw(s) 9 and/or 7, and then re-positioned into a different tibial or femoral tunnel sites. The knee is again put through a full range of motion and the movement of pointer 5 again monitored in relation to the isometer body 4. This re-positioning procedure for the movable stylus 2 and/or the fixed stylus 1 and the corresponding guide pins 10 and 8 may be repeated several time altering the site methodically around the originally chosen site and if after having repeated this process numerous times with no success, only then is the tibial site at Gerdy's tubercle considered for adjustment and the femoral sites are retested against a secondly selected Gerdy's tubercle site.

Once pointer movement 5 is acceptably small indicating that isometry has been determined, the set screws 7 and 9 are loosened to release the guide pins 8 and 10 from their corresponding styli 1 and 2, and the isometer is then removed, leaving the guide pins 8 and 10 in place. The femoral pin is drilled through the femoral cortex exiting medially and sufficiently proximal to avoid the femoral tunnel of the previously reconstructed ACL. The tibial pin is advanced distally and medially in order to avoid the tibial tunnel (if present in an accompanying ACL reconstruction) until it exits the skin.

The guide pins are then used to direct placement of the drill, but once the tunnels are created the guide pins are removed and the reconstructive surgery is then completed.

In a different variation of the methodology of using the isometer, guide pins are first inserted into the femur and tibia at the proposed positions of the femoral and tibial tunnel sites. The styli of the isometer are then aligned with and placed over the guide pins and the guide pins are locked in position using a locking mechanism so that the guide pins are firmly held in position during movement of the knee. The knee is then moved through a normal flexion range of motion as described above and the subsequent steps of the methodology are the same.

Referring now to FIG. 2 and elements of FIG. 1, an enlarged view of elongated cannulated body 4, can be seen, with openings 13 for encasing slider rod 3 of FIG. 1 and opening 14 in the first end of body 4 for the cannulated fixed stylus 2 that is attached transversely through the first end of the isometer body d and further has a corresponding fixed-stylus guide pin 8 that slides vertically within the cannulated fixed stylus 2, and thus through the first end of the isometer body 4. The cannulated elongated body may also have a side window opening 12 for viewing movement of the pointer 5 along the slider rod 3.

FIG. 3A shows a top view and FIG. 3B shows side view of slider rod 3, and typical dimensions that may be used in accordance with the present invention. The slider rod 3 includes slot 20 so that the slider rod 3 slides around fixed stylus 2 within cannulated body 4. The hole 21 of the slider rod 3 allows the movable stylus 1 to be inserted and held in position relative to the slider rod 3 during flexing of the knee.

It should be understood by one of ordinary skill in the art that each stylus, may include a sharp point for insertion into a bone and that the isometer could be used without guide pins. In such an embodiment, the markings made in the bone by the stylus would be used for either subsequent insertion of a guide pin and used in conjunction with a cannulated drill or the markings could be used with a manual or power drill. It should also be understood that the use of guide pins is preferred since guide pins provide additional precision in the positioning of the femoral and tibial tunnels, since the cannulated drill drills at precisely the position of the guide pin whereas a manual drill may walk away from the marking and therefore provide a less precise tunnel location.

The present invention provides both a surgical device and a reproducible and standardized method for determining lateral rotary isometry in combined ALR/ACL reconstruction. The isometer of the present invention also essentially eliminates the technique-intensive and time-consuming problems inherent in “free-hand” determinations of lateral rotary isometry.

Although various exemplary embodiments of the invention have been disclosed, it should be apparent to those skilled in the art that various changes and modifications can be made that will achieve some of the advantages of the invention without departing from the true scope of the invention. These and other obvious modifications are intended to be covered by the appended claims.

1. An isometer for use during lateral augmentation surgery for antero-lateral rotatory instability for use in determining lateral isometry, the isometer comprising:
a body elongated about a first axis;
a slider which slides relative to the body about the first axis;
a stylus attached to the body; and
a stylus attached to the slider.

2. The isometer according to claim 1, wherein the elongated body has a channel into which the slider slides.

3. The isometer according to claim 1, wherein the body has a side with an opening that exposes the slider.

4. The isometer according to claim 3, wherein the slider includes a pointer which is visible through the opening on the side of the body.

5. The isometer according to claim 4, wherein the body has indicia for measuring position of the pointer attached to the slider.

6. The isometer according to claim 1, wherein each stylus includes a point capable of insertion into a bone.

7. The isometer according to claim 6, wherein the point of each stylus is formed by a guide pin and each stylus comprises a housing formed to receive the guide pin.

8. The isometer according to claim 6 wherein each sheath includes a restraint for holding the guide pin in the housing when the guide pin is inserted into a bone.

9. The isometer according to claim 7 wherein after insertion into a bone, the restraints are releasable such that the isometer may be removed while leaving each guide pin in a bone.

10. The isometer according to claim 8 wherein each restraint includes a set screw which can be tightened to secure the guide pin.

11. An isometer for measuring accuracy of femoral and tibial tunnel placement to prevent knee instability, the isometer comprising:
an elongated body having a first end and a second end, and a top side and a bottom side;
a slider rod having a first end and a second end whereby the rod slides horizontally within the body and also having a longitudinal slot running nearly the length of the rod;
a cannulated fixed stylus having an open-end entry and an open-end tip attached transversely to the first end of the isometer body;
a fixed-stylus guide pin that slides vertically within the cannulated fixed stylus;
a fixed-stylus set screw that immobilizes and releases the fixed-stylus guide pin within the fixed stylus;
a cannulated movable stylus having an open-end entry and an open-end tip attached transversely to the first end of the slider rod;
a movable-stylus guide pin that slides vertically within the cannulated movable stylus;
a movable-stylus set screw that immobilizes and releases the movable-stylus guide pin within the movable stylus; and
a pointer encircling the slider rod that slides with the slider rod but whose position along the rod can also be adjusted manually.

12. An isometer according to claim 11 wherein the slider rod is threaded on the first end to receive the movable stylus.

13. An isometer according to claim 11 further comprising a cannulated handle attached to the first end of the isometer body, aligned such that the fixed-stylus guide pin can be directed through the cannulated handle, through the isometer body, through the slider rod, and into the fixed stylus.

14. An isometer according to claim 11 further comprising an elongated body with a side opening for viewing pointer movement.

15. An isometer according to claim 11 wherein the isometer is used to evaluate variation in the distance between two points during in vitro range of motion testing in order to determine the excursion values as they are related to isometric tunnel placement for augmentation graft insertion into the points yielding rotary and lateral stability corresponding to a pre-determined acceptable flexibility/stability standard.

16. An isometer according to claim 11 wherein the elongated isometer body includes an etched ruler for objectively monitoring and measuring pointer movement.

17. A method for determining a position of isometry using the device of claim 11, comprising the steps of:
a) directing guide pins into the fixed and movable styli;
b) advancing the fixed-stylus guide pin until the tip protrudes beyond the fixed stylus tip;
c) advancing the movable-stylus guide pin until the tip protrudes beyond the movable stylus tip;
c) tightening the fixed- and movable-stylus set screws thereby immobilizing the guide pins within the styli;
d) positioning the immobilized guide pins over previously identified lateral reconstruction tunnel sites such that either the fixed stylus is positioned over i) a femoral tunnel site and the movable stylus is positioned over a tibial tunnel site or ii) the fixed stylus is positioned over a tibial tunnel site and the movable stylus is positioned over a femoral tunnel site;
e) driving the guide pins into the bone tunnel sites;
f) flexing and extending the knee slowly, while maintaining the isometer position;
g) monitoring the movement of the pointer in relation to the isometer body as the knee is flexed and extended;
h) if movement of the pointer is unacceptably large, re-positioning the styli and repeating the above steps b) through g) as necessary until pointer movement is acceptably small;
i) if the pointer movement is acceptably small, loosening the fixed and movable set screws to allow removal of the isometer; and
j) leaving the guide pins in place.

18. A method for determining a position of isometry according to claim 17 further comprising the step:
identifying tunnel sites such that when the isometer is positioned in the tunnel sites movement of the pointer is 3 mm or less.

19. A method for determining a position of isometry according to claim 17 further comprising the step:
identifying tunnel sites such that when the isometer is positioned in the tunnel sites movement of the pointer is 2.5 mm or less.

20. A method for determining a position of isometry according to claim 17 further comprising the step:
identifying tunnel sites such that when the isometer is positioned in the tunnel sites movement of the pointer is 2 mm or less.

21. A method for determining a position of isometry to prevent rotary instability of a knee using an isometer having a body with a slider that moves relative to the body, the body attachable to a first bone at a point of attachment and the slider attachable to a second bone at a point of attachment, the method comprising:
attaching the isometer to the first bone and to the second bone; moving the knee through a range of motion;
if movement of the slider relative to the elongated body is less than a maximum, drilling a tibial and femoral tunnels at the points of attachment.

22. The method according to claim 21 wherein if movement of the slider is greater than the maximum, repositioning at least one point of attachment of the isometer;
moving the knee through a range of movement;
drilling the tibial and femoral tunnels at the points of attachment if the movement of the slider is less than the maximum.

23. The method according to claim 21 wherein the body includes a stylus for receiving a guide pin; and
the slider includes a stylus for receiving a guide pin.

24. The method according claim 23, wherein the step of attaching the isometer to the first bone and the second bone includes;
inserting a first guide pin through the stylus associated with the body into the first bone; and
inserting a second guide pin through the stylus associated with the slider into the second bone.

25. The method according to claim 24, further comprising:
locking the first guide pin in the stylus associated with the body; and
locking the second guide pin in the stylus associated with the slider.

26. The method according to claim 21, wherein drilling includes:
seating a cannulated drill on a guide pin at the point of attachment of the first bone;
drilling a first tunnel at the first position;
seating the cannulated drill on a guide pin at the point of attachment of the second bone;
drilling a second tunnel at the second position;

27. The method according to claim 24, further comprising removing the isometer while leaving the guide pins affixed to the first and second bones.

28. The method according to claim 21 wherein drilling includes measuring the distance that a pointer which is attached to the slider moves using indicia of distance that is marked on the body.

29. A method for determining a position of isometry to prevent rotary instability of a knee using an isometer having a body with a slider that moves relative to the body, the isometer further having a stylus attached to the slider and a stylus attached to the elongated body, the method comprising:
inserting a first guide pin at a desired location of a femoral tunnel;
inserting a second guide pin at a desired location of a tibial tunnel;
placing one of the styluses over the first guide pin;
placing the other stylus over the second guide pin;
moving the knee through a range of motion;
if movement of the slider relative to the elongated body is less than a maximum, drilling the tibial and femoral tunnels at the location of the first and second guide pins.

30. The method according to claim 29, wherein if the movement of the slider is greater than the maximum, repositioning at least one of the guide pins to a new location.

31. The method according to claim 30, further comprising:
inserting the guide pins into the tibia and the femur;
moving the knee through a range of movement; and
drilling the tibial and femoral tunnels using the guide pins if the movement of the slider is less than the maximum.
